(19) United States
(12) Patent Application Publication
(10) Pub. No.: US 2007/0287923 A1
(43) Pub. Date: Dec. 13, 2007

(54) Wrist plethysmograph

(76) Inventors: Charles Adkins, Earlsville, VA (US); David Gerdt, Faber, VA (US); Martin Baruch, Charlottesville, VA (US)

Correspondence Address: PARKER INTELLECTUAL PROPERTY LAW OFFICE, 536 PANTOPS CENTER, # 234, CHARLOTTESVILLE, VA, 22911, US

(21) Appl. No.: 11/803,643

(22) Filed: May 15, 2007

Related U.S. Application Data
(60) Provisional application No. 60/800,521, filed on May 15, 2006.

(51) Int. Cl. A61B 5/021

(52) U.S. Cl. 600/485

(57) ABSTRACT

A pulse monitoring plethysmograph system for establishing a history of the pulses of the user over an extended period of time, comprises a housing, a piezoelectric sensing element mounted within said housing, and fixed to the housing, a force transmitting member positioned to cause said piezoelectric sensing element to flex in response to an external force and to generate a current, and a transimpedance amplifier. The transimpedance amplifier converts the current generated by the flexing of the piezoelectric element into a voltage signal and an analog to digital converter converts the voltage signal into digital data. A digital memory storing member is provided for storing the digital data and establishing a history of data over an extended period of time.

### CROSS-REFERENCE TO RELATED APPLICATIONS

This application claims the benefit of provisional patent application 60/800,521 for Wrist Plethysmograph, filed May 15, 2007, the disclosure of which is incorporated herein as though recited in full.

### GOVERNMENT INTEREST STATEMENT

ONR (Office of Naval Research) NO-0014-04-C-0204

### FIELD OF THE INVENTION

The present invention relates generally to a system for measuring an arterial pulse, and more particularly to a means by which arterial pulse can be continuously measured with a noninvasive device that makes direct mechanical contact with the user's wrist.

### BACKGROUND

### Background of the Invention

Beat by beat blood pressure is the missing piece of information in lie detection and psychological response to immediate stressors. Post-traumatic stress disorders might be manageable by simply quantifying the accumulated stress that a Marine is experiencing. Stress has known negative effects on the cardio vascular system. Heart rate variability (HRV) seems to be a window into observing the accumulation of stress or pending illnesses. From what the literature illustrates, measuring heart rate or HRV or any other useful parameter is more difficult during the wakened period of the day when there are often additional stressors. At night, measurement of HRV is easier and may be more useful because there is only one activity.

The pressure pulse, the mechanical representation of the blood flowing in the artery, is best detected at the classic pressure points that are well known and whose locations are widely published in the literature. At these points the artery is close to the surface of the skin so that with application of light constrictive pressure, the pulsations caused by the heartbeat can be sensed mechanically as pulsations in the constrictive force. At some points, the artery is so close to the surface that slight pulsations can even be observed on the surface of the skin. On many people, the radial artery produces such a disturbance at the wrist pressure point and provides an opportunity for pulse measurement at that sensing point.

### SUMMARY

The present invention relates to a plethysmograph for measuring heart rate variability (HRV) and other HR derived parameters and storing monitored data within a wristwatch-like device for later download using a connection to a PDA or PC, via a connection such as USB. In one example of a military application, the mitigation starts with an observation made by a corpsman who notices, for instance, that a Marine has a deteriorated HRV pattern. This observation will occur as the watches of all the Marines are periodically reviewed by the Corpsman. Mitigation in its simplest form might amount to giving the Marine a couple of days off in a safer, more relaxed setting until the HRV returned to normal values. The wristwatch-like device is thus used to correlate accumulated stress or pending sickness with HR derived parameters.

According to a first broad aspect of the present invention, there is provided a wrist-watch like device that includes a piezo sensing element.

According to a second broad aspect of the invention, there is provided a piezo sensing element that is caused to flex in response to expansion and contraction of the diameter of the user's wrist.

According to another broad aspect of the invention, there is provided a pulse monitoring system for establishing a history of the pulses of the user over an extended period of time.

According to still another broad aspect of the invention, there is provided a system for externally sensing the disturbance at the wrist pressure point, in which the disturbance causes a piezoelectric sensing element to generate an electrical current.

According to a further broad aspect of the invention, there is provided a transmitting system for transmitting pressure pulses that are sensed at a wrist pressure point by a piezoelectric sensing element to an external receiver.

According to a further broad aspect of the invention, there is provided a piezoelectric element supported on a substrate member that is preferably an elastically deformable metal, such as brass and steel. The piezoelectric fragility toward flexing is minimized by virtue of being bonded to the deformable substrate member.

According to still another broad aspect of the invention, there is provided a wrist plethysmograph having a piezoelectric member supported on a deformable substrate member and housed within a Faraday cage which is mounted by a strap to the user's wrist. The strap of the wrist plethysmograph is relatively inelastic and upon swelling of the user's wrist, the strap pulls a back plate component toward the wrist and the back plate member drives a force transmitting element against the piezoelectric member and it substrate carrier member. The piezoelectric is caused to flex and generates a measurable current.

According to still a further board aspect of the invention, a system for externally sensing the disturbance at the wrist pressure point is employed, in which the disturbance is caused by the pulsing of an artery or arteries in the user's wrist. The resulting wrist expansions and contractions bend a piezoelectric sensing element causing it to generate an electrical current.

According to still a further board aspect of the invention, a system for externally generating a train of waves corresponding to arterial pressure pulses in a user's wrist is provided in which the pressure pulses bend a piezoelectric sensing element causing it to generate an electrical current. A transimpedance amplifier is employed to transform the current signal to a required voltage signal. The voltage is continuously zeroed out, resulting in low sensitivity to electromagnetic fields, motion artifacts, thermo gradients, etc.

According to still a further board aspect of the invention, a wrist plethysmograph is contained within a wristwatch-like structure, and an electrical signal is wirelessly transmitted to a remote receiver.

According to still a further board aspect of the invention, a wrist plethysmograph is contained in a wristwatch-like structure, and an electrical signal is generated by the application of a force to a piezoelectric disk, as by bending, flexing, or the like. The signal data is stored within a memory component within the watch, and transmitted to a remote receiver either by a direct wire connection, or wirelessly. Bluetooth signal transmission or the like can be used for the wireless signal transmission to a nearby PC, PDA, smart phone, or the like, where further analysis can take place and where-alarms can be triggered. Additionally, or alternatively, phone messages, email messages, or the like can be sent with, or without the wears knowledge or assistance, to care givers, emergency medical services, Internet sites, or other interested party or parties.

### BRIEF DESCRIPTION OF THE DRAWINGS

The invention will be described in conjunction with the accompanying drawings, in which:

FIG. 1 is a schematic side view of a plethysmograph on a wrist, in accordance with an embodiment of the present invention;

FIG. 2 is a schematic partial side view of a plethysmograph in a housing and responding to increased pressure, in accordance with an embodiment of the present invention;

FIG. 3 is a schematic side view of a plethysmograph on a wrist, without applied expansive pressure, in accordance with another embodiment of the present invention;

FIG. 4 is a schematic side view of another embodiment of a plethysmograph on a wrist, with applied expansive pressure, in accordance with another embodiment of the present invention;

FIG. 5 is a schematic illustration of the transducing of the expansive force on a strap to a bending force on a piezoelectric element, and the consequential generation of a current by the piezoelectric element, in accordance with another embodiment of the present invention;

FIG. 6 is a schematic illustration of a piezoelectric element within a Faraday shield or cage, in accordance with an embodiment of the present invention;

FIG. 7 is a circuit diagram of a transimpedance circuit, in accordance with an embodiment of the present invention;

FIG. 8 is a graph illustrating the frequency gain response curve from a transimpedance circuit, in accordance with an embodiment of the present invention;

FIG. 9 is a circuit diagram of a voltage amplification circuit;

FIG. 10 is a graph illustrating the frequency gain response curve from a circuit such as that of FIG. 9;

FIG. 11 is a circuit diagram of a standard, non-inverting amp,

FIG. 12 is a graph illustrating the frequency gain response curve from a circuit such as that of FIG. 11.

FIG. 13 is a graph illustrating a pulse wave generated by a plethysmograph in accordance with an embodiment of the present invention; and

FIG. 13A is a graph illustrating a pulse wave train generated by a plethysmograph in accordance with an embodiment of the present invention.

### DETAILED DESCRIPTION

It is advantageous to define several terms before describing the invention. It should be appreciated that the following definitions are used throughout this application.

Definitions

Where the definition of terms departs from the commonly used meaning of the term, applicant intends to utilize the definitions provided below, unless specifically indicated.

For the purposes of the present invention, the term “disk” refers to all polygonal shapes, from a three sided polygon (triangle) to an infinite sided polygon (circle), and is inclusive of oblong configurations, e.g., ellipses, elongated rectangles, and the like.

For the purposes of the present invention, the term “flexing” refers to the physical deforming, (e.g. bending, twisting, stretching, etc.) of a piezo element that causes said piezo element to generate a current. Bending of the piezo element can be, for example, on the order of microns to tens of microns in response to a force of tens to hundreds of Newtons.

For the purposes of the present invention, the term “fulcrum” refers to a point or support about which a piezoelectric sensing element flexes.

For the purposes of the present invention, the term “inelastic” refers to being resistant to dimensional change. A strap must be sufficiently flexible to comfortably conform to the shape of the user's wrist, but must not stretch under the load that is being applied. Inelastic is used synonymously with “intractable” and “non-extensible”, to indicate a high resistance to change, in particular, resistant to a change in length under the force produced by the expanding of the wrist due to arterial pulses. Examples of inelastic/intractable/non-extensible materials that can be used for the strap are silk, rayon, Kevlar, Twaron, and aramid fibers. Kevlar® is the DuPont Company's brand name for a particularly light but very strong synthetic fiber consisting of long molecular chains produced from poly-paraphenylene terephthalamide. Twaron® is a para-aramid and is used in automotive, construction, sport, aerospace, military and industry applications, e.g., “bullet-proof” body armor. Spider silk is a fiber spun by spiders. Spider silk is a remarkably strong material and its tensile strength is comparable to that of high-grade steel. Spider dragline silk has a tensile strength of roughly 1.3 GPa, as compared to a tensile strength for one form of steel of 1.65 GPa. However, spider silk is much less dense than steel having a ratio of tensile strength to density that is perhaps 5 times better than steel. Spider silk is as strong as Aramid filaments, such as Twaron or Kevlar. Aramid fibers are a class of strong, heat-resistant synthetic fibers. They are used in aerospace and military applications, for “bullet-proof” body armor fabric, and as an asbestos substitute. The name is a shortened form of “aromatic polyamide”. They are fibers in which the chain molecules are highly oriented along the fiber axis, so the strength of the chemical bond can be exploited. High Tenacity Rayon is a modification of “regular rayon” to provide exceptional strength (two times that of HWM rayon). High tenacity rayon is primarily found in tire cord and industrial end uses. It may be finished, chemically coated or rubberized for protection from moisture and potential loss of dimensional stability and strength during use.

For the purposes of the present invention, the term “piezoelectricity” refers to ability of crystals and certain ceramic materials to generate a voltage in response to applied mechanical stress. The piezoelectric effect is reversible in that piezoelectric crystals, when subjected to an externally applied voltage, can change shape by a small amount. (For instance, the deformation is about 0.1% of the original dimension in piezo element.) The effect finds useful applications such as the production and detection of sound, generation of high voltages, electronic frequency generation, microbalance, and ultra fine focusing of optical assemblies.

For the purposes of the present invention, the term “piezo element” refers to any material that has capability of generating piezoelectricity.

For the purposes of the present invention, the term “plethysmograph” refers to an instrument that measures variations in the size of an organ or body part on the basis of the amount of blood passing through or present in the part.

For the purposes of the present invention, the term “PZT” refers to a piezo element, as for example, one of lead zirconate titanate, a material that shows a marked piezoelectric effect as well as any other electroceramic that contains the properties necessary achieve the results set forth herein.

For the purposes of the present invention, the term “watch” refers to a wrist plethysmograph that is housed in a wrist watch-like device that can optionally include the features of a standard watch, chronograph, and the like.

For the purposes of the present invention, the term “transimpedance amplifier” refers to a circuit for converting current input into voltage output. A typical situation is the measuring of current using instruments having voltage inputs. A current-to-voltage converter is a circuit that performs current to voltage transformation. In electronic circuitry operating at signal voltages, it usually changes the electric attribute carrying information from current to voltage. The converter acts as a linear circuit with transfer ratio k=V_{OUT}/I_{IN} [V/mA] having dimension of resistance. The active version of the circuit is also referred to as a transresistance or transimpedance amplifier.

### DESCRIPTION

The disclosed plethysmograph can track beat-by-beat blood pressure—even during sleep without waking the patient—measuring peripheral blood pressure. There are important differences between peripheral and central blood pressure in the treatment of hypertension, especially resistant hypertension. The disclosed device uses Pulse Decomposition Analysis (PDA) for a complete understanding of the pulse wave structure and is based on verified physical theory. The device measures heart and breathing rates, arrhythmias, and inspiratory effort through direct observation of pulsus paradoxus, heart rate variability, cardiac output, and other parameters. These measurements can enable the device to detect and locate aortic aneurisms and can be useful as a screening device for aneurisms, saving about $5,000 in hospital costs. It can also be useful for monitoring arterial stiffness, general arterial health, apnea as a screening device, drug therapy compliance, efficacy studies for drugs, CPAP compliance and efficacy, asthma screening or warning device, and as a health protector for first responders. The device can automatically monitor heart rate, determine heart rate variability, and store irregular heart rate patterns.

Since the disclosed plethysmograph can quantify inspiratory effort, it can be used as a hospital admission standard for children who may be suffering from asthma attack, enabling the separation of emotional symptoms from asthma symptoms. At home the disclosed plethysmograph can measure the severity of an asthma attack or even warn of an impending attack. As a monitoring device, inspiratory effort could be an important device for the diagnosis and treatment of many respiratory problems including chemical exposure, environmental asthma, and insults to the lungs or trachea. Glucose levels could also be detected indirectly from a combination of parameters that the disclosed plethysmograph supplies. Nocturnal hypoglycemia is particularly important to warn or wake a patient before the effects of a glycemic coma disable the patient. Apneas and hypopneas during sleep could also be recorded and, since heart and breathing rates are recorded, central apnea could be diagnosable at home or in the field. Because of the measurement of inspiratory effort, obstructive sleep apnea could be diagnosable.

The plethysmograph provides a means by which arterial pulse can be continuously measured using an inexpensive, simple and noninvasive device with direct mechanical contact to the wrist or other part of the body to be monitored. The wrist, at the radial artery palpation area, provides an accurate monitoring area due to the slight swelling of the wrist and arm in response to the passage of the pulse. Although this effect can be weak and not be sensed adequately, the movement of the tissue surrounding the palpation area is often visible to the eye.

In light of the accuracy obtained through monitoring the radial artery palpation area, the disclosed invention will be primarily described through examples using a watch. The disclosed watch can also provide standard watch functions and, as most people wear watches anyway, the monitoring device would provide no change to normal patterns or habit. Watch type features, controlled by user buttons, can be incorporated, as for example stop watch and countdown timer(s), temperature measurement, compass, radiation level detector, altimeter, etc. It will be evident, however, to those skilled in the medical arts, how to adopt the teachings to monitor other areas of the body.

The disclosed watch uses the imperceptible swelling of the wrist that occurs as the arterial pulse travels down the arm and is terminated at the fingertips. This termination is also imperceptible and dies out without notice unless a finger is damaged due to trauma when pulsitile throbbing occurs. The band and watchcase are snuggly wrapped around the wrist. When the imperceptible pulse arrives it is observed by a piezo element and creates a free charge as the swelling of the wrist bends the element. A current, reacted by the movement of this charge, is transformed by the transimpedance amplifier into a voltage which represents the change in pulse pressure with time. Provided the system is well shielded and pre-amplified, integration of this signal reproduces the pulse pressure waveform familiar in textbooks. The current is differentiated to get pulse integration. Without the fast recovery provided by the transimpedance amplifier, settling times can be five to ten seconds. It also eliminates the piezo element's large sensitivity to thermal transients, which are substantial in a body worn device that is exposed to the environment on the side opposite the wrist.

To monitor these palpitations, a mechanical strap surrounds the wrist at its palpation point over the radial artery. If the strap is worn with light tension, the pulse at the palpation point can cause a variation in the tension of the mechanical strap that is mechanically transferred to a piezoelectric sensing element housed in a container similar to a conventional wristwatch. The strap is a narrow band or strip of material, as for example, a watch band that overlies the volar aspect or anterior of the wrist.

A physical transfer element, mounted between the surface of the skin and the piezoelectric sensing element transfers the variations in contact pressure to the piezo sensing element. The pressure fluctuations on the piezo element can be sensed as current or voltage changes.

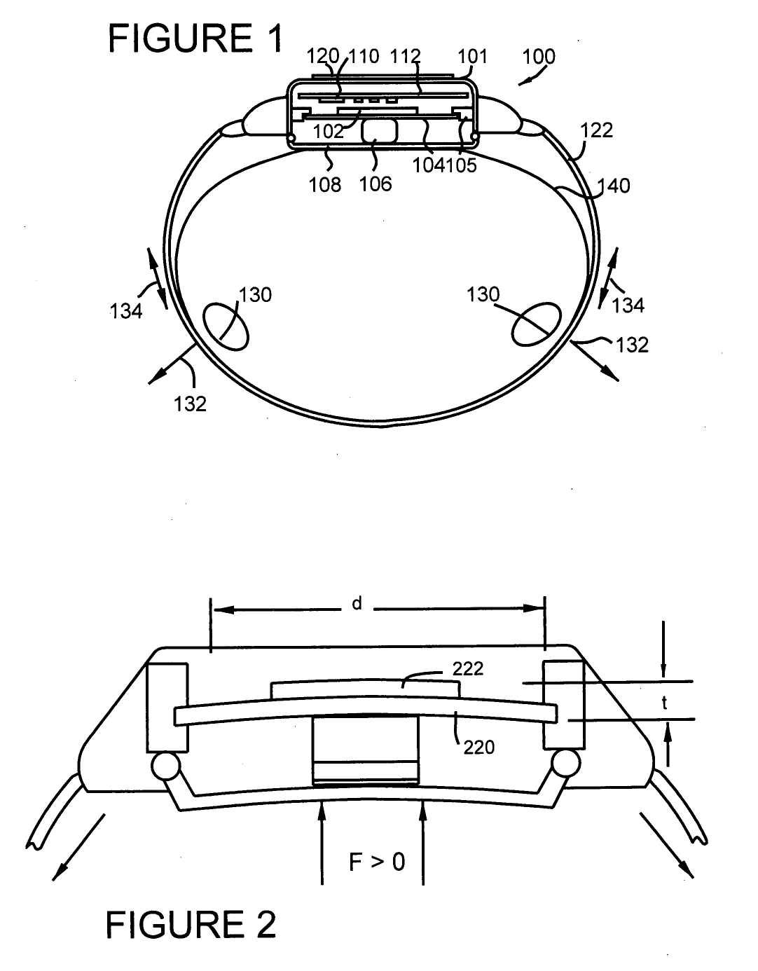

A substantial amount of information can be obtained through the monitoring of heart rate and blood pressure. As the disclosed watch can measure, record and analyze both separately and in combination, the heart rate and blood pressure, a vast amount of information can be obtained about the user. Examples of information derived from the disclosed watch are:
Heart rate: The differentiated signal from the piezo element is composed mostly of a noise floor interrupted by a rising waveform representing the pulse. The rate is the reciprocal of the inter-beat interval. The average heart rate over a circadian period has been used to specifically diagnose many mental illnesses.
Inter beat-interval: The time between the successive pulse wave forms in the signal from the piezo element.
Heart rate variability: HRV is computed from the record of inter-beat intervals.
Abnormal patterns: Unusual patterns in raw signal such as arrhythmias or missing beats could be recognized by embedded software so that examples could be saved in memory or they could be analyzed locally with storage of the analysis.
Blood pressure: Because the pulse waveform is rather high quality, it contains enough information, when integrated and analyzed, to provide an estimate of blood pressure. The features necessary for pulse decomposition analysis, (PDA®), are visually observable.
HR*BP: With heart rate and blood pressure, the actual workload on the heart can be calculated. Heart rate alone estimates the heart workload much more crudely. More importantly, there have been studies showing that the aerobic-to-anabolic threshold can be reliably detected by a combination of heart rate and blood pressure (called the double product break-point, DPBP. This method; while perhaps not as accurate as a VO₂ max test device, could be valuable for maximally efficient training. As shown in FIG. 1 the plethysmograph is illustrated in the form of a wrist watch like device indicated generally as 100. The device 100 is secured to a user's wrist 140 through use of a strap 122 that is positioned to be in contact with the radial artery 130. The two arteries shown correspond to the general artery location on a right hand and a left hand rather than to two arteries. The radial pulse is sensed on the underside of the wrist 140, just below the thumb.

The device 100 has an outer casing 101 that houses the components of the plethysmograph and also serves as a Faraday cage. The outer casing 101 shields the high impedance components from electrical interference, protecting the piezoelectric sensing element and its amplifier from surrounding changing electromagnetic fields.

A piezoelectric element 102 is supported by a substrate member 104 which is fixed to a non-conductive ring member 105, that can be formed of any insulating material, such as fiberglass or other polymeric material. The piezoelectric element 102 is constructed in a classical bending configuration and more specifically is geometrically designed as a bending disk. An advantage of using circular disks, such as found in piezoelectric buzzers, is their ubiquitous availability and minimized cost of construction due to a very high production volume. However, other configurations of disks can also be used as defined above and will be evident to those skilled in the art.

The substrate member 104 is centrally supported by a fairly rigid support member 106. Upward movement of the support member 106, produced by the upward movement of the back face 108 of the watch like member 100, causes a flexing of the piezoelectric element 102. The support member 106 functions like a fulcrum about which the substrate member 104 and the piezoelectric element 102 are flexed. In accordance with this embodiment the contact pressure of the strap 122 with the wrist 140 produces a tensile force that is opposed by the contact pressure at the back face 108 of the outer casing 101 of the sensor housing. If the pressure is directly applied to the piezoelectric element 102, the element 102 can easily be broken by the application of too much pressure. The piezoelectric element 102 is preferably protected by an elastomeric substrate support 104, limiting the deflection of the piezoelectric element and preventing breakage. The limitation of the movement of the piezoelectric element 102 is referred to as “snubbed”.

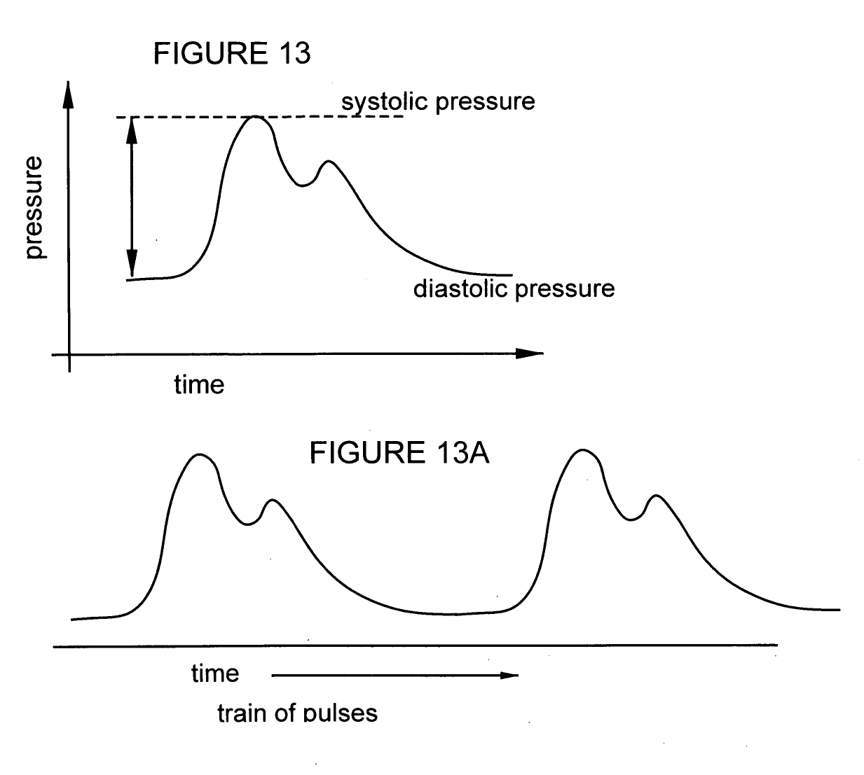

The current generated by the flexing of the piezoelectric element 102 is amplified by the transimpedance amplifier 110 carried by the circuit board 112. The transimpedance charge amplifier 110 is used as a current to voltage converter in an inverting configuration to output the differential or time derivative of the electrical signal from the piezo element. The rapid processing of the electrical signal is essential to the generating of the train of pulse pressure waves, as shown for example, in FIG. 13A. In a preferred embodiment, the transimpedance amplifier 110 is directly connected to the piezoelectric element 102 so that it measures the current (i) produced by changes in stress (dδ/dt) or strain (dε/dt) on the piezoelectric element 102 and not the voltage which results from the stress or strain directly. In the preferred embodiment, the integration of the derivative of the pulse signal can be performed digitally by several techniques. In an alternate embodiment, the pulse can be integrated by adjusting the time constant of the transimpedance amplifier to below the fundamental frequency of the pulse.

Because the disclosed plethysmograph contains a local area network radio, other sensors, such as body temperature or smart bandages, could be implemented within the disclosed system. A host of external, self-powered sensors could be added to the disclosed system depending on their usefulness with the plethysmograph acting as the hub enabling several of sensors to be part of a customized automatic monitoring system. Examples of these include oximetry at the ear, toe, or other places, ECG, EMG, EEG, skin salinity, a variety of oral chemical detectors, and temperature. Audio monitoring of lung sounds using a matrix of microphones to form a picture of the lungs could be generated on a display that locates and identifies sites of pneumonia, projectile damage, and other lung problems.

The disclosed device can store the history of treatment and medical records of the patient. These records and medical history can be viewed or altered by a PC or PDA. Due to Moore's Law, devices can store data can store can retain a patient's entire treatment history and all relevant personal information even if the period is over weeks, months, or years.

A transimpedance amplifier converts current input to voltage output. The piezo element converts tensile stress in the piezo element to displacement of electrical charge Q. Thus Q=k₁₃*F. Since dQ/dt−k₁₃*dF/dt and dQ/dt=i then the transimpedance amplifier's output voltage is proportional to the time derivative of force applied to the sensing element. If the pulse waveform spectrum is decomposed into a set of sine waves (the Fourier Transform) then the fundamental definition, dQ/dt=ω*cos(Ωt) reveals the obvious fact that the derivative of the set of sine functions falls at 20 dB/decade to zero as ω approaches D.C. Thus, if the current representing the movement of charge between shorted electrodes is measured instead of the open circuit voltage between them, D.C. blocking is intrinsic in the measurement and no external capacitor is needed.

Although possible to use, the use of a voltage amplifier is far less advantageous than a transimpedance amplifier 110 because the pulse signals have a very low frequency of about 0.1 Hz. A 35 mm piezo element has about 0.02 μF capacitance and the voltage while measuring a pulse is nominally about 1 volt. At 1 Hz, 1 volt on the capacitance of the piezoelectric element causes about 0.1 μamp to flow. Therefore it would require a voltage amplifier with a gain=1 to be about equal to a transimpedance amplifier with R_{f}=10 MOhms.

The back face 108 is pulled tight against the wrist 140 of the user by the watch band or strap 122 as the wrist 140 swells with each pulse. The back face 108 acts like a piston and pushes the transfer member 106 against the substrate 104 and the piezoelectric element 102, to which it is bonded. The piezoelectric element 102 bends, producing a tiny varying charge separation, which is a current signal. The strap 122 is non-elastic and thus resists expanding and contracting under the pulse pressure indicated by arrows 132. The strap 122 does not expand or contracting in the direction indicated by arrows 134, and consequently, the swelling of the wrist is accommodated by the plethysmograph by driving the casing back face 108 outwardly toward the piezoelectric member 102. Upon passage of the pulsation the substrate member and the tendency of the casing back to flatten, drive the watch casing back 108 to its zero force configuration.

By clamping the piezoelectric voltage to zero, the configuration produces a very linear response and removes the sensitivity to environmental influences and keeps the electrical signal centered around zero (0) volts, where it can be suitably amplified without regard to slowly varying voltage offsets that might be derived from changes in temperature, pressure, or light intensity. It should be noted that the electrical response is directly the time differentiated pulse and must be integrated to get the normally recognized pulse response.

The mechanical strap 122 that couples the radial pulse to the piezoelectric element 102 acts as a mechanical filter and special attention must be given to the physical characteristics of that element. The straps disclosed herein must be inelastic in order to accurately couple the radial pulse to the piezoelectric element. If the strap is highly compliant, that is, very elastic, the pulsative signal will be attenuated in the elastic distortion of the strap rather than transferring the signal to the piezoelectric element 102. If the strap 122 is very stiff, it will not conform to the surface of the skin and will not respond well to the pulse. The material selected for the construction of the strap must be skin conformable while capable of transferring the signal from the wrist 140, or palpation point or points, to the piezoelectric element 102, with out attenuating the signal to a level that obviates or negates the sensitivity of the piezoelectric element 122. That is, the signal that is transferred to the sensor must be sufficient to produce the required level of current.

An example of a material that will enable the flexing of the piezoelectric element 102 is rayon or silk cloth. However other materials that are stiff in the plane of the fabric, and highly compliant, i.e., has a high tensile strength, but has a low resistance to flexural and/or torsional forces, out of the plane of the fabric can be used.

The extraction of the pulse into an electrical form enables the analyzing of the pulse through algorithms. The wrist plethysmograph 100 does not merely count the time frequency of the user's pulse, but also senses the patterns of the pressure variations. Using the data from the plethysmograph piezoelectric element 102 various physiological parameters can be monitored, including heart rate, blood pressure, breath rate, augmentation index (indication of the hardness of arteries), heart rate variability, and other physiological cycles.

FIG. 2 shows the flexure of piezoelectric element 222 in response to the Force F which is greater than zero. The substrate 220 is an elastically deformable metal, such as brass, steel, and the like. The lower the thickness “t” of the piezoelectric element 22 and substrate 220, the greater the sensitivity, and the greater the delicacy of the piezoelectric element 222. The greater the diameter “d”, the greater the sensitivity and the greater the delicacy of the piezoelectric element 222.

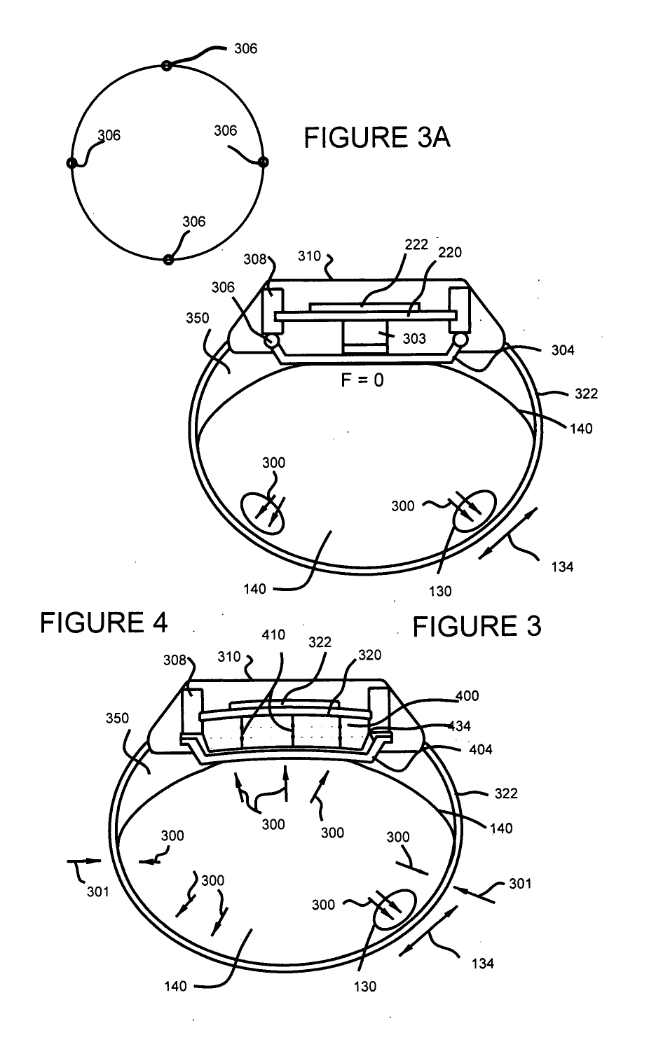

FIG. 3 shows the piezoelectric element 222 under a zero force. The elastically deformable member 220 is peripherally fixed by the support component 308, which is turn is fixed to the “watch case” 310. It should be understood that the term “watch case” is employed because of the physical appearance, of the plethysmograph is like that of a wrist watch. While the plethysmograph can readily house a watch, in particular, a digital watch, there is nonfunctional requirement for the plethysmograph to actually look like a watch.

The compliant anchors 306, as illustrated in FIG. 3A secure the back face 304 to the plethysmograph housing 310, while permitting the case back face 304 to move slightly. The user's wrist is indicated by reference numeral 140, and a radial artery is indicated as 130. The expansion of the radial artery 130 at the surface of the wrist produces a force indicated by arrows 300. The two arteries shown correspond to the general artery location on a right hand and a left hand rather than to two arteries. The radial pulse is sensed on the underside of the wrist, just below the thumb.

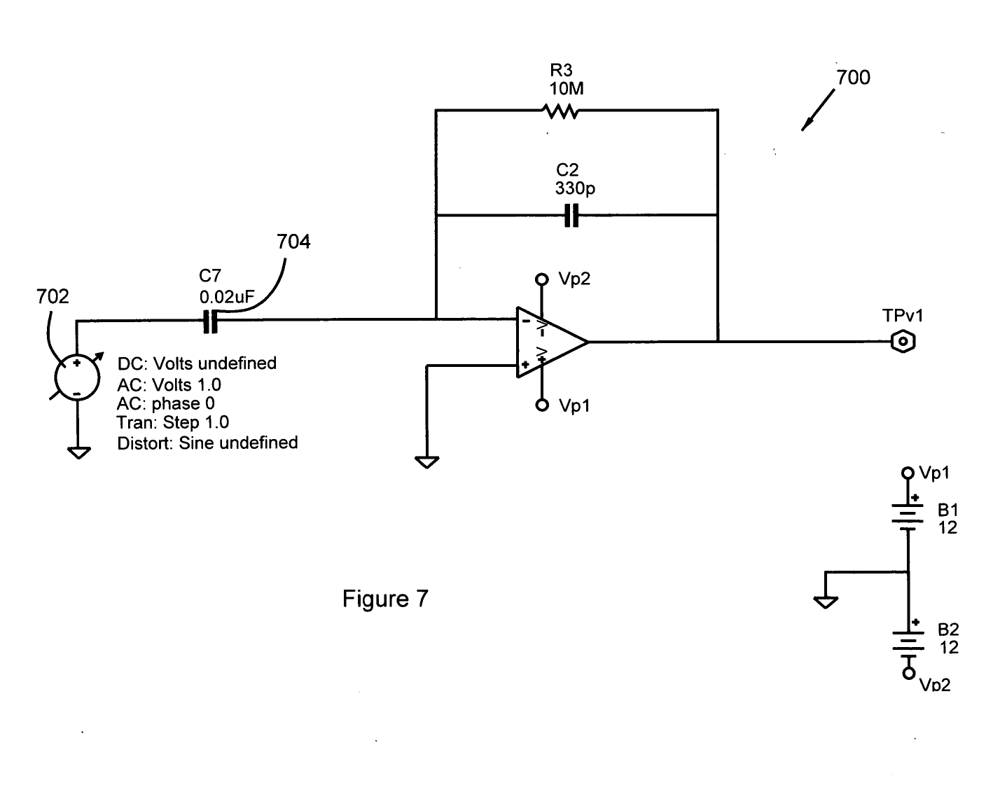

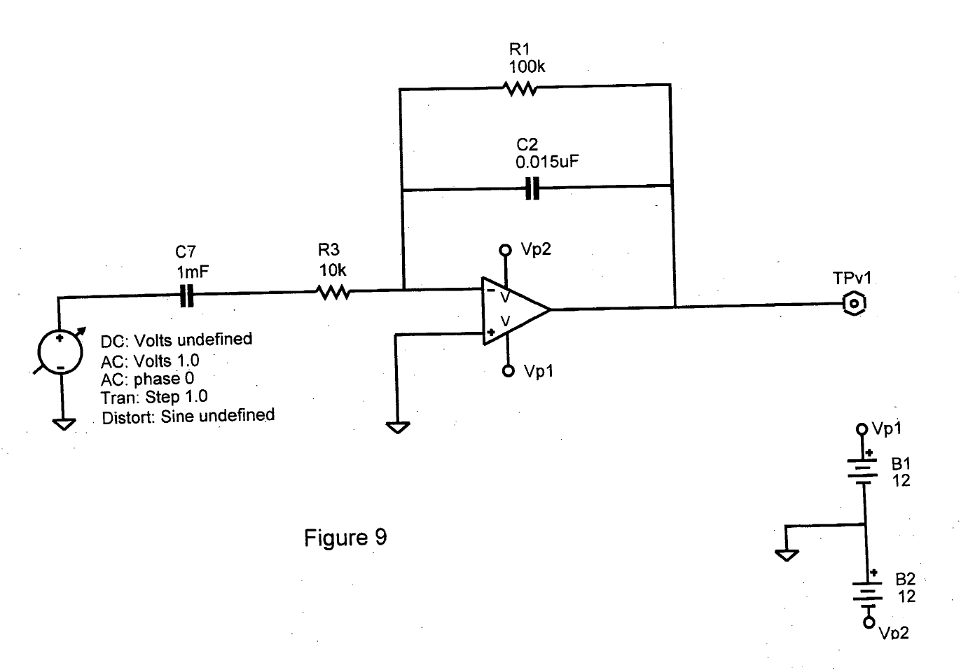

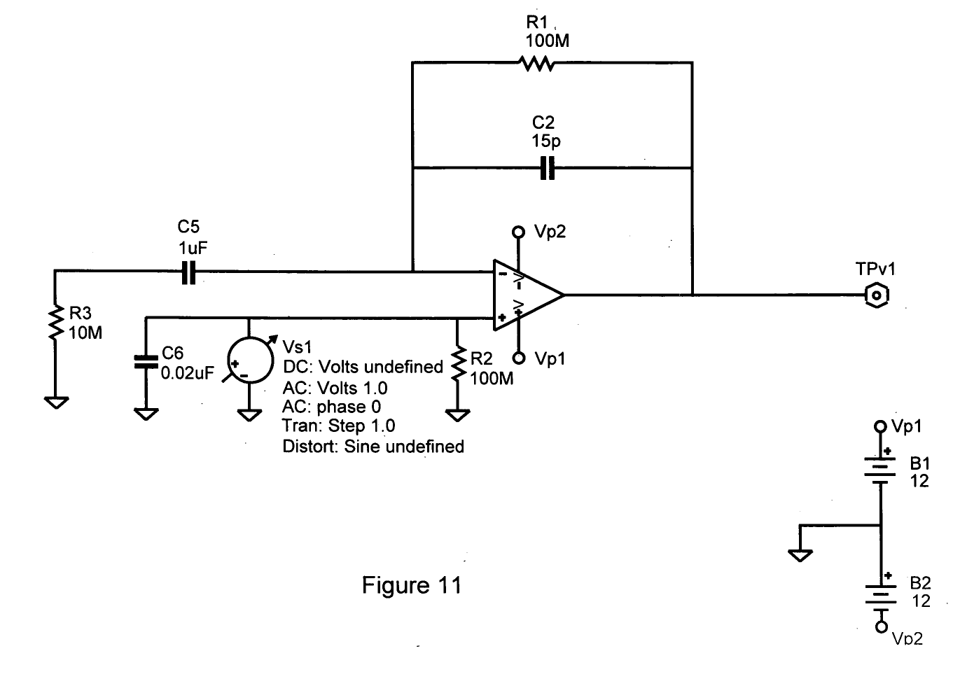

The expansion force indicated by the arrows 300 apply a force to the strap 322, in the direction indicated by the arrow 134. The inelasticity of the strap 322 results in the force 300 being applied to the back face 304 of the housing 310. The term “bottom face” refers the face of the watch that is in contact with the posterior of the wrist. When the resultant force applied by the transfer member 303 to the piezo element 222 is greater than zero, as indicated in FIG. 2, the piezoelectric bending disk 222 flexes and generates a current signal that is transformed into a voltage signal, using a circuit such as illustrated in FIG. 7, FIG. 9, and FIG. 11. The transfer member 303, being essentially inelastic, forces the piezoelectric element 222 to flex, as if about a fulcrum point or support.

The strap 322 is seen to closely conform to the shape of the user's wrist 140, and essentially the only gap between the strap 322 and the wrist is in the region 350, proximate the housing 310.

FIG. 4 shows an alternate embodiment in which the physical force transfer medium is a fluid 400, in a pressurized chamber formed by a gasket or seal member 434 adjacent to the housing case 310 back face 404. The fluid applies a pressure 410 uniformly across the surface of the substrate member 320. Since the substrate member 320 is peripherally fixed to the housing at support 308, the substrate member 320 is force to flex primarily at its center, causing a flexing of the piezoelectric disk member 322. Although the forces that must be supported by the mechanical components are the same, the protection of the piezoelectric element is much easier in the fluid filled element. As described heretofore, the strap 322 is in contact with the wrist 140 except are areas 350 which are adjacent to the housing 310. The force 300 applied as the wrist 140 swells causes the back facing 404 to transfer the pressure through the substrate 320 to the piezoelectric member 322. The opposing force exerted by the strap 322 is indicated by arrow 301. Alternative, air can be used as the fluid element.

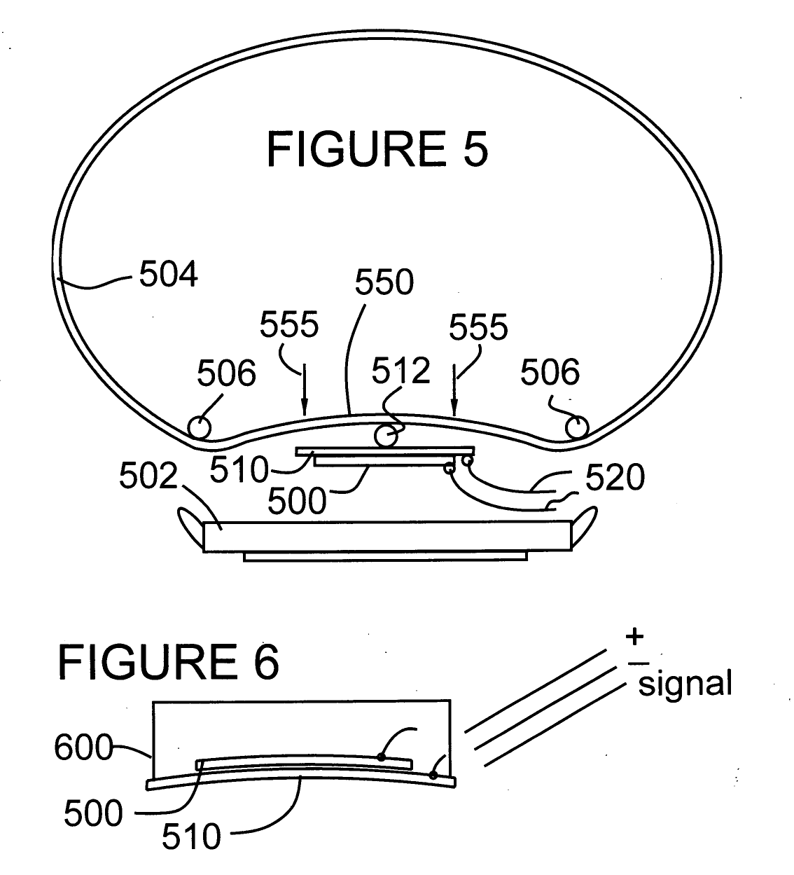

In the schematic illustration of an embodiment of the invention of FIG. 5, a strap 504 is forced to elongate as the wrist swells, thereby causing the reverse arcuate region 550 to straighten. The term “reverse arcuate” is employed to signify a curvature that is concave relative to the convex curvature of a user's wrist. The resultant outward movement of the region 550 exerts a force in the direction indicated by arrows 555. The fulcrum member 512 is driven against the substrate member 510 causing the piezoelectric member 500 to flex and generate a current. The various components are housed within the casing 502 that is shown separated from the strap region 504 and the anchor elements 506. The electrical leads 520 connect the piezoelectric element 500 to the transimpedance amplifier (not shown) to convert the current into voltage.

FIG. 6 shows the flexed substrate member 510 and piezoelectric member 500 within a Faraday cage 600. The transimpedance circuit is also within the Faraday cage to further exclude electrostatic environmental noise.

FIG. 7 is an example of a simple transimpedance circuit in which the capacitor 704 and current source 702 are in series and are equivalent to the piezoelectric current source. In the circuit of FIG. 7, the voltage is forced to zero at the inverting input and the transformation is of the current, rather than an amplification of the voltage. At 100 mHz, its reactance becomes significant relative to the 100M-Ohm feedback resistance and the low frequency break causes the effective gain of the circuit to fall away towards D.C.

The capacitance of the piezo electrodes appears in parallel to the effective voltage source in the voltage amplifier model and its reactance, greater than 100K at the highest frequency in our passband, is therefore ignored.

The frequency regime used in the disclosed plethysmograph covers the resting breathing fundamental at the low frequency extreme to the upper frequencies contained in the heartbeat. The passband therefore is about 100 milliHz to about 60 Hz.

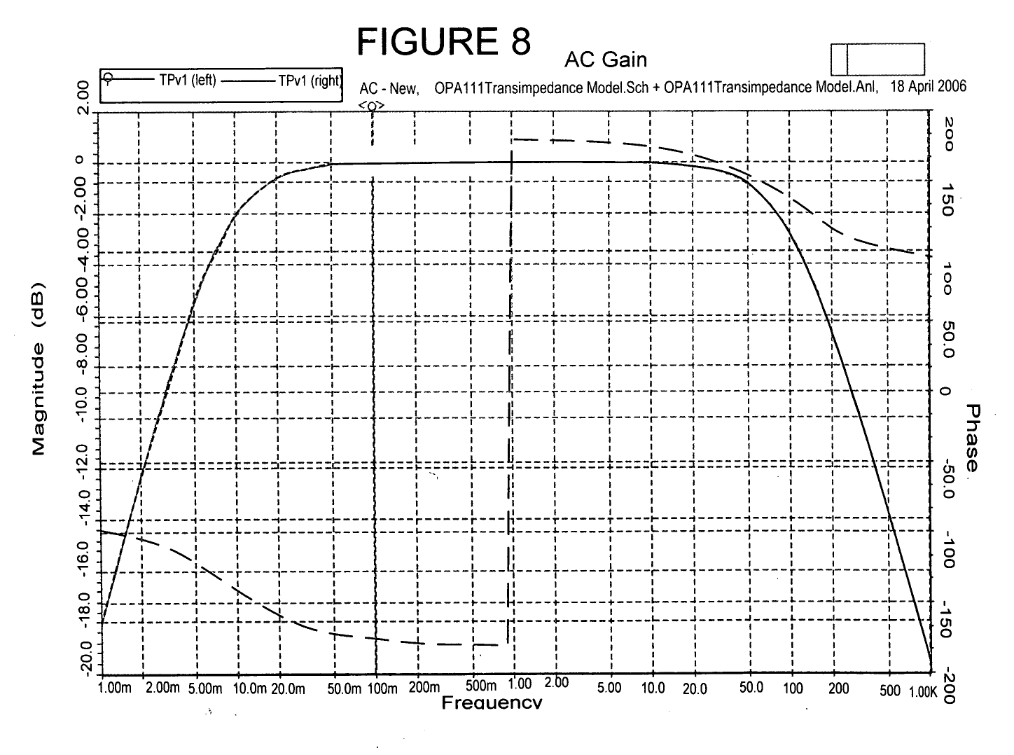

FIG. 8 corresponds to the output of the transimpedance circuit of FIG. 7, and illustrates the desired output for an adult human.

A conventional D.C. coupled voltage amplifier will pass all of the frequencies from D.C. to the upper break point determined by R_{f} and C_{f} (½*π*R*C) Since the signal level is so high from the piezo element, very little gain can be used when the open circuit voltage is measured, in the range of about 1× to 10×.

The voltage amplifier signal output is breathing or pulse, directly, without signal processing. The signal contains, however, thermal inputs and motion artifacts to the constrained piezo element. The artifacts are large and the signal randomly drifts several volts from the zero reference.

Because of this artifact the voltage amplifier needs to include a blocking capacitor in the input to remove sensitivity to voltage signals approaching D.C. For our uses, the low frequency break point must be below the breathing fundamental at 0.1 Hz and the capacitor needed to accomplish this is very large.

As can be seen through the examples illustrated herein, there are a number of advantages to using a transimpedance amplifier circuit.
1. Since the output current from the piezo element represents the time derivative of the signal, it is always centered around zero volts and maximum gain can be used to set the system Noise Figure without fear of the signal clipping at the power supply rails.
2. By clamping the voltage across the capacitive elements of the piezo sensor, back emf which could retard the motion of charge in the circuit does not develop and maximum linearity is obtained.
3. The Transimpedance Amplifier Circuit gets rid of the very large input capacitance which is required to remove low frequency thermal drift from the measured signal.
4. By clamping the voltage across the inputs to zero, the circuit offers a very low impedance to ElectroMagnetic Influence from external sources. The high impedance input line offered by the voltage amplifier circuit is, on the other hand, a very good antenna.
5. The parts used in the circuit are minimal in number and small in size.
6. Response to motion artifacts is very fast, that is, there is a rapid recovery from motion artifacts, thus negating the negative impact of motion artifacts.

The gain curves are specifically tailored through electronic components to exclude signals below the human breathing rate and signals above the significant harmonics of the heart rate. It should be understood that the amplifiers electronic components can change depending on whether the system is used on a neonate, child, adult, or animal. Because the heart and breathing rates are somewhat different from each. Spurious low frequency signals, external signals from changing electrostatic fields, slowly moving temperature gradients, light level responses, and the like, are rejected by the amplifier by increasing the signal to noise ratio of the system. Higher frequency signals, usually due to motion or environmental vibrations are similarly rejected and result in higher signal to noise ratios.

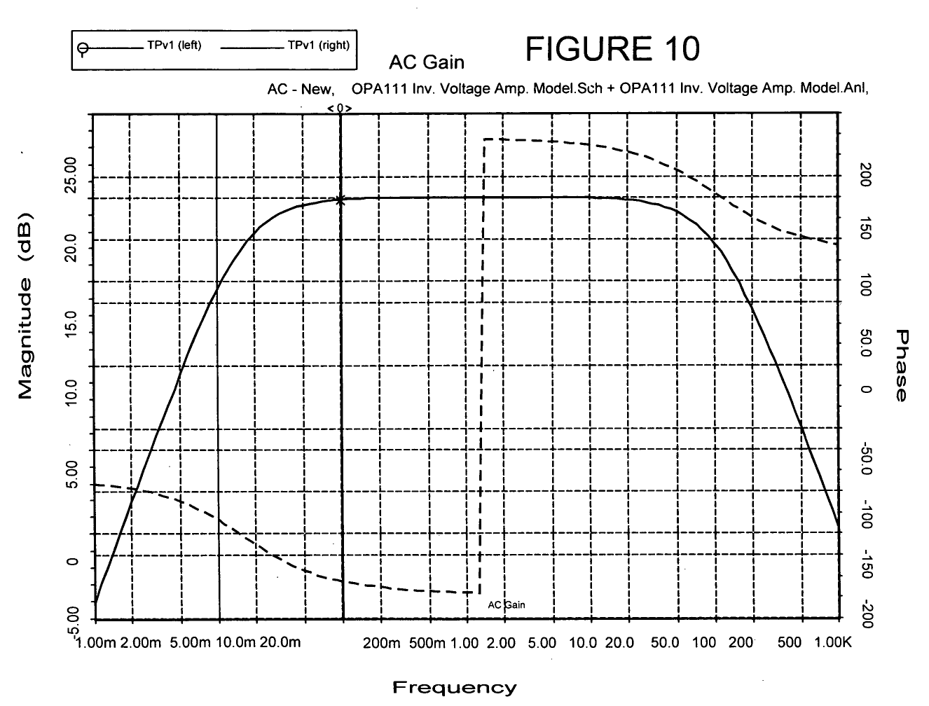

FIG. 9 illustrates a less preferred inverting amplifier circuit that requires components that are impractically large for installation into a watch-sized case to produce the required low frequency gain. FIG. 10 is the frequency response graph of the output of the circuit of FIG. 9.

The circuit of FIG. 11 is a less preferred non-inverting amplifier. To produce a gain, R₃ must be very large, and consequently there would be little signal.

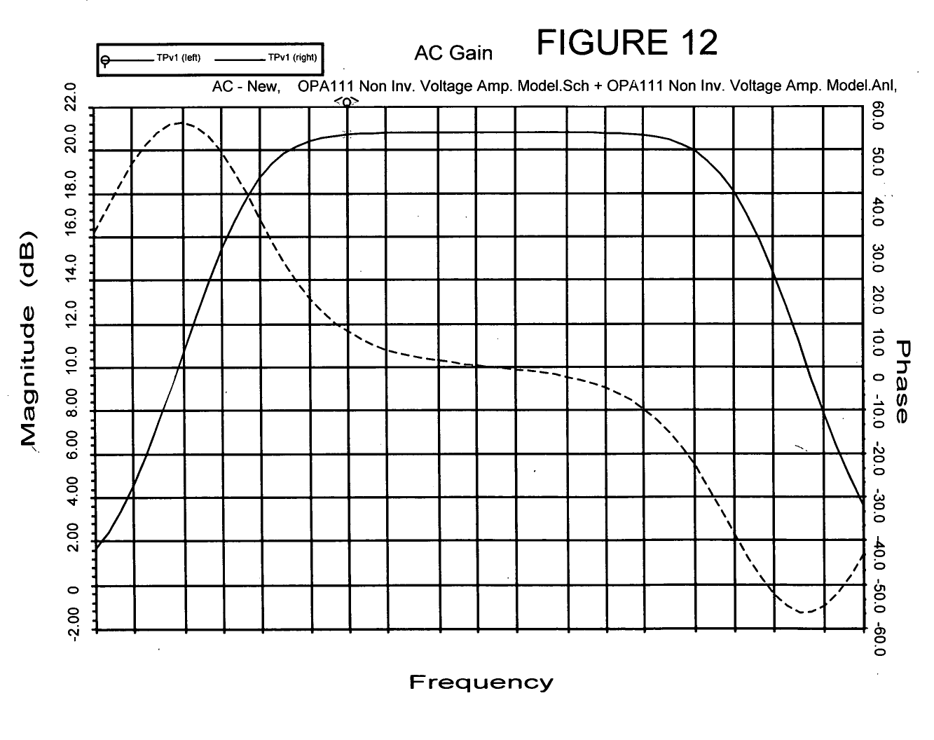

FIG. 12 is the frequency response graph of the signal from a non-inverting amplifier circuit of FIG. 11.

FIG. 13 illustrates a pressure curve that is generated by the integration of the piezoelectric element current. The information derived is not limited to the time interval between the peaks systolic pressure, (heart rate), but also includes information derived by the slopes of the curves, the various peaks and valleys between the systolic pressure and the diastolic pressure, and the arrival times, peak amplitudes and peak widths of the constituent pulses that sum to the familiar arterial wrist pulse.

FIG. 13A illustrates a train of pulse pressure waves caused by the swelling of the wrist. The plethysmograph is thus seen to provide a history of the pressure waves over an extended period of time. Depending upon the capacity of the battery used to power the plethysmograph, the time period can be days, weeks, months, etc.

Additional features can be added to the plethysmograph through software or integration of additional mechanical devices. Examples of these additions are:
Pulse decomposition analysis for BP and BP*HR. Software can be provided to prepare the signal for a PDA, and programming the PDA.
A USB/Bluetooth watch dongle for cell phone communication can be employed in the present system. This device, the size of a watch fob, will attach to the watch using the mini USB connector. The watch dongle can have its own high capacity coin cell so as not to over use the watch coin cell. This radio enabled system could be useful in the case of multiple casualties or as input to an automatic triage system when corpsmen are overburdened. It is not necessary that the radio be Bluetooth, but such radios can talk to cell phones, which are becoming more common than the wristwatch. Military personnel with cell phones or other RF communications systems could use the watch for downloading simple instructions or plans to be displayed on the watch LCD, uploading information stored in the watch, and synchronization of time.
Actigraphic features can be added to the system with the addition of a MEMS accelerometer or by cleverly using the piezoelectric element. The piezoelectric element already generates a noise floor that should change with activity level. If the activity level changes enough between sleep and normal activity, then the watch essentially has an actigraph built in. If it was not possible to use the inherent properties of the piezo element, then a MEMS accelerometer can be added to the watch board.
Automated treatment for casualties: Use radio controlled infusion pumps to administer drugs in dosages set in software in code. Some drugs could be administered on an as needed basis determined by the software within the disclosed plethysmograph.

### EXAMPLE I

Screening device for aortic aneurysms: Two subjects can be observed using the disclosed plethysmograph on both wrists. One patient has a thoracic aneurysm and the other patient has an abdominal aneurysm. In both cases disclosed plethysmograph detected and located the aneurysm. The disclosed plethysmograph is able to locate aneurysms because the pulse waveform is, a record of reflected systolic pulses that originate at different places and arrive at different times. Normally, there are only four reflected pulses, but, in these special cases, the aneurysm produces an extra pulse. As expected, no reflections are observed on a patient with a repaired abdominal aneurysm.

### EXAMPLE II

Automatic monitoring of spinal cord injured patients: HRV can be used as a pre-symptomatic indication of sepsis in neonates. HRV could be used for pre-symptomatic warnings of frequent urinary track infections, sepsis, or other impending illness. The disclosed plethysmograph can be tested on a number of SCI patients who often have one or more UTIs per month in order to monitor the reduction in HRV that precedes the symptoms of illness.

As stated heretofore other areas of the body can be used for monitoring. While most patients will have at least one hand, both will be,missing in some cases. In these instances, an alternative area, such as the upper brachial area, can be used for the monitoring. With the exception of the means for attachment, plethysmograph remains the same, except that algorithms must be adjusted for differences in anatomy and point of observation. Since there are pulse palpation sites on the forehead, feet, legs and neck, these places would in many cases provide all the presently obtained parameters with only changes in algorithm constants and coupling devices to fit the new anatomical geometry.

There are other reasons for alternate pulse sites. Like the waveform differences from a damaged aorta, insults to the femoral artery could be observable in the pedal pulse. An important use could be at the carotid artery, where the waveform changes dramatically as perfusion is reduced in the brain.

### BROAD SCOPE OF THE INVENTION

While illustrative embodiments of the invention have been described herein, the present invention is not limited to the various preferred embodiments described herein, but includes any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to examples described in the present specification or during the prosecution of the application, which examples are to be construed as non-exclusive. For example, in the present disclosure, the term “preferably” is non-exclusive and means “preferably, but not limited to.”

In this disclosure and during the prosecution of this application, means-plus-function or step-plus-function limitations will only be employed where for a specific claim limitation all of the following conditions are present in that limitation: a) “means for” or “step for” is expressly recited; b) a corresponding function is expressly recited; and c) structure, material or acts that support that structure are not recited. In this disclosure and during the prosecution of this application, the terminology “present invention” or “invention” may be used as a reference to one or more aspect within the present disclosure. The language present invention or invention should not be improperly interpreted as an identification of critically, should not be improperly interpreted as applying across all aspects or embodiments (i.e., it should be understood that the present invention has a number of aspects and embodiments), and should not be improperly interpreted as limiting the scope of the application or claims. In this disclosure and during the prosecution of this application, the terminology “embodiment” can be used to describe any aspect, feature, process or step, any combination thereof, and/or any portion thereof, etc. In some examples, various embodiments may include overlapping features. In this disclosure, the following abbreviated terminology may be employed: “e.g.” which means “for example”.

What is claimed is:

1- A blood pressure sensing device comprising a housing, a piezo sensing element mounted within said housing, said housing comprising a face plate and a wrist posterior contact member, said piezo sensing element being fixed to said face plate, and means to cause said piezo element to flex in response to an external force on said wrist posterior contact member, wherein said external force is the pressure of arterial blood.

2- The blood pressure sensing device of claim 1 further comprising a strap member secured to said face plate, said strap being sufficiently flexible to conform to the volar aspect of the wrist of the user and non-extensible.

3. The blood pressure sensing device of claim 2, wherein said strap is formed from a member selecting from the group comprising silk, rayon, Kevlar, Twaron, aramid fibers, and combinations thereof.

4- The blood pressure sensing device of claim 2, wherein said piezo sensing element is positioned within said housing such that it is caused to flex in response to expansion and contraction of the circumference of the user's wrist and further comprising a transimpedance amplifier, said transimpedance amplifier converting the current generated by flexing of said piezo sensing element into a voltage signal.

5- The blood pressure sensing device of claim 4, wherein said means to cause said piezo sensing element to flex in response to an external force on said wrist posterior contact member is a force transferring member positioned between said wrist posterior contact member and said piezo sensing element, whereby movement of said wrist posterior contact member causes the flexing of said piezo sensing element in response to an external force on said wrist posterior member.

6- The blood pressure sensing device of claim 5, wherein said piezo sensing element is supported on a substrate member that is elastically deformable, whereby said piezo sensing element's fragility toward flexing is minimized by virtue of being bonded to said deformable substrate member.

7- The blood pressure sensing device of claim 5, wherein said housing is a Faraday cage and wherein said wrist posterior contact member is insulated from said housing.

8- The blood pressure sensing device of claim 5, wherein said strap member is sufficiently inelastic that upon swelling of the user's wrist, said strap pulls said housing against the posterior of the user's wrist and said wrist posterior contact member drives a force transmitting element against said piezo sensing member and its substrate carrier member, causing said piezo sensing element to flex and generate a measurable current directly related to a user's blood pressure.

9- The blood pressure sensing device of claim 8, wherein said metal is selected from the group comprising brass, steel, and alloys thereof.

10- A pulse monitoring plethysmograph system for establishing a history of the pulses of the user over an extended period of time, comprising
a housing,
a piezoelectric sensing element mounted within said housing, said piezoelectric sensing element being fixed to said housing,
a force transmitting member positioned to cause said piezoelectric sensing element to flex in response to an external force and to generate a current,
a transimpedance amplifier, said transimpedance amplifier converting the current generated by flexing of said piezoelectric element into a voltage signal,
an analog to digital converter for converting said voltage signal into digital data, and digital memory storing member for storing said digital data and establishing a history of data over an extended period of time.

11- The pulse monitoring system of claim 10, wherein said data represents user arterial pressure waves.

12- The pulse monitoring system of claim 10, wherein the voltage across the capacitive elements of the piezoelectric sensing element are clamped whereby maximum linearity is obtained.

13. The pulse monitoring system of claim 10 having a circuit wherein the voltage the inputs are clamped to zero, whereby the circuit offers very low impedance to electromagnetic influence from external sources.

14. The pulse monitoring system of claim 10 wherein the output current from said piezoelectric sensing element represents the time derivative of the signal, and is centered around zero volts, whereby maximum gain can be used to set the system noise figure without fear of the signal clipping at the power supply rails.

15. The pulse monitoring system of claim 10, wherein said history of data includes data from pulse decomposition analysis software.

16. The pulse monitoring system of claim 10, further comprising actigraph software for monitoring user activity level changes.

17. The pulse monitoring system of claim 10, further comprising drug administering infusion pumps, said drugs being administered on an as needed basis determined by software within said plethysmograph.

18. The pulse monitoring system of claim 10, wherein said system further comprises wireless data transmitter and wherein said drug administering infusion pumps are wirelessly controlled.

19. The pulse monitoring system of claim 10, further comprising a signal data storage memory component within said housing, and a data transmitter for transmitting data to a remote receiver by a direct wire connection, or wirelessly and wherein said remote receiver has data analysis software.

20. A method of continuous non-invasive blood pressure monitoring, comprising strapping a plethysmograph to at least one of a user's wrists, physically altering a piezoelectric sensing element in response arterial wrist pulse producing wrist expansions and contractions, whereby said piezoelectric element generates an electrical current, converting said current into a voltage signal by means of a transimpedance amplifier circuit, and generating digital data from said voltage signal, said data corresponding to blood pressure wave forms.

21. The method of claim 20, further comprising screening for aortic aneurysms comprising strapping a plethysmograph to each of a user's wrists, monitoring the pulse wave form generated by said plethysmograph, recording reflected systolic pulses that originate at different places and arrive at different times, and monitoring said reflected systolic pulses for an extra pulse or pulses.
